(19) United States
(12) Patent Application Publication
(10) Pub. No.: US 2011/0144205 A1
(43) Pub. Date: Jun. 16, 2011

(54) Blood Glutathione as a Biomarker for Screening Asymptomatic Patients at Risk for Heart Failure

(75) Inventors: Thibaud Damy, Creteil (FR); Philippe Le Corvoisier, Creteil (FR); Catherine Pavoine, Creteil (FR); Francoise Pecker, Creteil (FR); Philippe Caramelie, Creteil (FR); Gisele Bonne, Paris (FR); Christophe Meune, Paris (FR)

(73) Assignee: INSERM (Institut National de la Recherche Medicale), Paris (FR)

(21) Appl. No.: 12/908,163

(22) Filed: Oct. 20, 2010

Related U.S. Application Data
(63) Continuation-in-part of application No. PCT/EP09/59242, filed on Jul. 17, 2009.

(30) Foreign Application Priority Data
Jul. 30, 2008 (EP) 08305438.7

(51) Int. Cl. A61K 31/197; C12Q 1/26; C12Q 1/37; G01N 33/53; A61P 9/04

(52) U.S. Cl. 514/563; 435/25; 435/23; 435/7.92

(57) ABSTRACT

The present invention relates to a method for screening an asymptomatic patient at risk for heart failure, said method comprising measuring the concentration of glutathione in a blood sample obtained from said patient.

### RELATED APPLICATIONS

The present application is a continuation-in-part of International Patent Application No. PCT/EP09/59242, which was filed Jul. 17, 2009, claiming the benefit of priority to European Patent Application No. 08305438.7, which was filed on Jul. 30, 2008. The entire text of the aforementioned applications is incorporated herein by reference in its entirety.

### FEDERALLY SPONSORED RESEARCH OR DEVELOPMENT

[Not Applicable]

### FIELD OF THE INVENTION

The present invention relates to a method for screening an asymptomatic patient at risk for heart failure. More particularly, the method of the invention comprises measuring glutathione in a blood sample obtained from said patient.

### BACKGROUND OF THE INVENTION

Heart failure is defined by the symptom complex of dyspnea, fatigue and depressed left ventricular systolic function (ejection fraction<35-40%), and is the ultimate endpoint of all forms of serious heart disease.

Despite considerable advances in treatment, heart failure remains associated with high morbidity and mortality. Heart failure has many causes and pathophysiological origins. For example, population at risk for developing heart failure include patients with ischaemic heart disease, previous myocardial infarction, atrial fibrillation, hypertension, diabetes, coronary artery disease or obesity, and elderly. Another important cause of heart failure is genetically-linked dilated cardiomyopathy (DCM), in which the most frequently encountered mutations are those in the lamin A/C (LMNA) gene. Prevalence and incidence rates of heart failure are growing, and it is presently a leading cause of hospitalization and death in developed countries, but will also likely become a major public health burden for developing countries.

Due to the seriousness of heart failure, identifying the individuals at risk of this syndrome is a necessity. Furthermore, screening and early diagnosis of patients at risk for heart failure is extremely desirable since this would make it possible to adapt a faster, easier and less expensive therapeutic monitoring.

For now, routine periodic assessment of LV function in all asymptomatic patients cannot be recommended. Finding biomarkers that are capable of detecting patients while still asymptomatic and possibly can predict those patients who will become symptomatic is an unmet need. A number of biomarkers for heart failure diagnosis and prognosis have been identified, which are from diverse biochemical groups and include brain natriuretic peptide (BNP), amino-terminal pro-brain natriuretic peptide (NT-pro BNP), norepinephrine, troponins, heart-type fatty acid binding proteins, myosin light chain-1, matrix metalloproteinases (MMPs), tissue inhibitors of metalloproteinases (TIMPs), C-reactive protein, tumour necrosis factor alpha (TNF-alpha), soluble tumour necrosis factor receptor-1 (STNFR1), soluble IL-2 receptor, and uric acid.

Blood levels of proinflammatory molecules, including sTNF and its receptors sTNFR-1 and -2, are elevated in heart failure patients of NYHA III to IV classes, and are highly predictive of adverse outcomes. However, neither TNF nor TNFR-1 or -2 tests do help to screening asymptomatic patients.

B-type natriuretic peptide (BNP) and the amino-terminal fragment of its precursor hormone (NT-pro-BNP) have also received considerable attention as potential screening tests for symptomatic and asymptomatic heart disease. They are secreted by the hemodynamically stressed heart mainly in response to myocardial stretch induced by volume overload. It is of general agreement that these peptides are markers of increased risk of death and hospitalization in symptomatic heart failure patients, and death in acute coronary syndromes. Accordingly, use of B-type natriuretic peptides as adjuncts to clinical diagnosis of heart failure is generally admitted. In asymptomatic patients, B-type natriuretic peptides could be normal or subnormal and the heart failure diagnosis could be misleaded. Furthermore, the low the specificity limits the clinical utility of these peptides as a general screening tool. In fact, increased plasma B-type natriuretic peptides is not unique to heart failure patients, but is also seen in patients with oedematous disorders, such as renal failure or ascitic liver cirrhosis, that lead to increased atrial tension or central blood volume.

Therefore the methods based current biomarkers as above described fail to screen the asymptomatic patients at risk for heart failure.

Inflammation and oxidative stress are key components in the pathophysiology and progression of heart failure, and are strongly associated with the disease severity.

The tripeptide glutathione (L-γ glutamyl-cysteinyl-glycine) does not only play a cardinal role in the maintenance of the cell redox status and defense against oxidative stress, but is also essential in many other cell functions, including cell survival. Recent studies have given evidence that glutathione deficiency determines the adverse effects of TNF, exacerbating sTNFR1-apoptotic and negative inotropic effects in isolated cardiomyocytes, and promoting cardiac remodelling in hypertensive and post-myocardial infarction heart failure rats. Furthermore it has been found that the left ventricle (LV) of end-stage heart failure patients is depleted by 54% in glutathione, compared with healthy control LV, and depletion in blood glutathione has been reported in advanced heart failure patients with dilated cardiomyopathy.

However, no study has yet examined whether blood glutathione level might be related with parameters characterizing heart failure, including the NYHA functional classification (New York Heart Association Functional Classification), cardiac function, assessed by standard echocardiography or Tissue Doppler Echography (TDE), and a blood soluble recognized biomarker of heart failure severity (e.g. sTNFR1).

### BRIEF SUMMARY OF THE INVENTION

[Not Applicable]

The invention relates to a method for screening an asymptomatic patient at risk for heart failure said method comprising measuring glutathione in a blood sample obtained from said patient.

The invention also relates to a method for classifying a patient at risk for heart failure, wherein said method comprises the steps of:

(i) measuring the concentration of glutathione in a blood sample obtained from said patient,

(ii) comparing the concentration of glutathione measured in step (i) to a reference value derived from the concentration of glutathione in blood samples from patients who are at particular stages of heart failure or to a control value derived from the concentration of glutathione in blood samples from healthy patients.

The invention relates to a kit for screening an asymptomatic patient at risk for heart failure where in said kit comprises means for measuring the concentration of glutathione in a blood sample obtained from said patient.

The invention also relates to the use of blood glutathione as a biomarker for screening asymptomatic patients at risk for heart failure.

Finally, the invention relates to glutathione precursors or drugs having the capability to restore glutathione level in the heart tissue, such as N-acetylcysteine, for the prevention of heart failure in a patient wherein said patient has been screened or classified according to the methods described above.

### DETAILED DESCRIPTION OF THE INVENTION

The inventors demonstrate that deficiency in blood glutathione is related with heart failure severity in cardiac patients. More particularly, the inventors have demonstrated that glutathione deficiency is related to altered cardiac function, and may be an interesting new biomarker for the early diagnostic of NYHA class I and II cardiac patients comprising LMNA-mutated patients. Therefore measuring blood glutathione deficiency in a patient may represent a screening test for patients at risk for heart failure. These findings also substantiate the indication of drugs having the capability to restore tissue glutathione, such as N-acetylcysteine, as a complementary therapy for the management of patients at risk for heart failure and displaying glutathione deficiency.

Diagnostic Methods and Kits:

The present invention relates to a method for screening an asymptomatic patient at risk for heart failure, said method comprising measuring the concentration of glutathione in a blood sample obtained from said patient.

As used herein the term “glutathione” has its general meaning in the art and refers to the total, oxidized and reduced forms of the tripeptide L-γ glutamyl-cysteinyl-glycine which, in its reduced form, has the formula of:

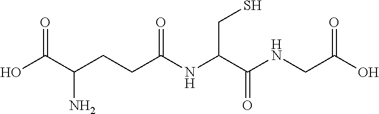

The term “blood sample” as used herein refers to a blood sample (e.g. whole blood sample, serum sample, or plasma sample) obtained for the purpose of in vitro evaluation.

As used herein, the term “patient” denotes a mammal, such as a rodent, a feline, a canine, and a primate. In a preferred embodiment of the invention, a patient according to the invention is a human.

According to a particular embodiment, the patient has been affected with a cardiac and/or vascular disease. For example, the patient may be diagnosed with a genetically linked cardiovascular disease, hypertension (high blood pressure), pulmonary hypertension, aortic and mitral valve disease (e.g. stenosis), aortic coarctation, coronary disorders, chronic arrhythmias (e.g. atrial fibrillation), cardiomyopathy of any cause, coronaropathy, valvulopathy or cardiac fibrosis. In another particular embodiment, the patient may be at risk for heart failure because of diabetes, obesity, aging, smoking, dyslipidemia, intoxication or a genetic disease. In another particular embodiment, the patient has a mutation of the LMNA gene coding for lamin A/C proteins, and hence is at risk for heart failure.

The term “asymptomatic patient” refers to a patient who has been classified as a NYHA class I patient. Functional classification of heart failure is generally done by the New York Heart Association Functional Classification (Criteria Committee, New York Heart Association. Diseases of the heart and blood vessels. Nomenclature and criteria for diagnosis, 6th ed. Boston: Little, Brown and co, 1964; 114). This classification stages the severity of heart failure into 4 classes (I-IV). The classes (I-IV) are:

Class I: no limitation is experienced in any activities; there are no symptoms from ordinary activities.

Class II: slight, mild limitation of activity; the patient is comfortable at rest or with mild exertion.

Class III: marked limitation of any activity; the patient is comfortable only at rest.

Class IV: any physical activity brings on discomfort and symptoms occur at rest.

Once the blood sample from the patient is prepared, the concentration of glutathione may be measured by any known method in the art.

For example, the concentration of glutathione may be measured by using standard enzymatic assay according to the method of Tietze F. et al. (Tietze F. Enzymic method for quantitative determination of nanogram amounts of total and oxidized glutathione: applications to mammalian blood and other tissues. Anal Biochem. 1969; 27:502-22.), as previously used by Bourraindeloup M. et al. (Bourraindeloup M, Adamy C, Candiani G, Cailleret M, Bourin M C, Badoual T, Su J B, Adubeiro S, Roudot-Thoraval F, Dubois-Rande J L, Hittinger L, Pecker F. N-acetylcysteine treatment normalizes serum tumor necrosis factor-alpha level and hinders the progression of cardiac injury in hypertensive rats. Circulation. 2004; 110:2003-9), and as recently updated by Rahman I; et al. (Rahman I, Kode A, Biswas S K. Assay for quantitative determination of glutathione and glutathione disulfide levels using enzymatic recycling method. Nat Protoc. 2006; 1:3159-65. Enzymatic assays utilize glutathione reductase for the quantification of glutathione). For example the sulfhydryl group of glutathione reacts with DTNB (5,5′-dithio-bis-2-nitrobenzoic acid, Ellman's reagent) and produces a yellow colored 5-thio-2-nitrobenzoic acid (TNB). The disulfide, glutathione that is produced, is then reduced by glutathione reductase to recycle the glutathione and produce more TNB. The rate of TNB production is directly proportional to this recycling reaction which in turn is directly proportional to the concentration of glutathione in the sample. Measurement of the absorbance of TNB at 405 or 412 nm provides therefore an accurate estimation of glutathione in the sample. Many enzymatic assays are commercially available from Alpco Diagnostics, Assay Designs/Stressgen Bioreagents, BioAssay Systems, BioChain, BioVision, Calbiochem, Cayman Chemical, GenScript Corporation, Immundiagnostik, Kamiya Biomedical Company, MBL International, Millipore Corporation, Molecular Probes (Invitrogen), Neogen Corporation, Novagen, Oxis International, Inc., Promega, Sigma-Aldrich, and Thermo Scientific Pierce Protein Research Products.

Other methods include reagents that selectively interact with glutathione, standard electrophoretic and immunodiagnostic techniques, including immunoassays such as competition, direct reaction, or sandwich type assays. Such assays include, but are not limited to, Western blots; agglutination tests; enzyme-labeled and mediated immunoassays, such as ELISAs; biotin/avidin type assays; radioimmunoassays; immunoelectrophoresis; immunoprecipitation, high performance liquid chromatography (HPLC), size exclusion chromatography, solid-phase affinity, fluorescent, colorimetric or radioactive probes that specifically interact with glutathione, etc.

In a particular embodiment, the methods of the invention comprise contacting the blood sample with a binding partner capable of selectively interacting with glutathione in said blood sample.

The binding partners may be fluorescent, colorimetric or radioactive probes that specifically interacts with glutathione such as bimanes, o-phtaldialdehyde (OPA), N-substituted maleimides, organometallics, etc. Such fluorescent probes may be commercially available from Calbiochem, Biovision Research Products, etc.

The binding partner may be an antibody that may be polyclonal or monoclonal. Polyclonal antibodies directed against glutathione can be raised according to known methods by administering the appropriate antigen or epitope to a host animal selected, e.g., from pigs, cows, horses, rabbits, goats, sheep, and mice, among others. Various adjuvants known in the art can be used to enhance antibody production.

Monoclonal antibodies against glutathione can be prepared and isolated using any technique that provides for the production of antibody molecules by continuous cell lines in culture. Techniques for production and isolation include but are not limited to the hybridoma technique; the human B-cell hybridoma technique and the EBV-hybridoma technique. Alternatively, techniques described for the production of single chain antibodies (see e.g. U.S. Pat. No. 4,946,778) can be adapted to produce anti-glutathione, single chain antibodies. Antibodies useful in practicing the present invention also include anti-glutathione fragments including but not limited to F(ab′)2 fragments, which can be generated by pepsin digestion of an intact antibody molecule, and Fab fragments, which can be generated by reducing the disulfide bridges of the F(ab′)2 fragments. Alternatively, Fab and/or scFv expression libraries can be constructed to allow rapid identification of fragments having the desired specificity to glutathione. For example, phage display of antibodies may be used. In such a method, single-chain Fv (scFv) or Fab fragments are expressed on the surface of a suitable bacteriophage, e.g., M13. Briefly, spleen cells of a suitable host, e.g., mouse, that has been immunized with a protein are removed. The coding regions of the VL and VH chains are obtained from those cells that are producing the desired antibody against the protein. These coding regions are then fused to a terminus of a phage sequence. Once the phage is inserted into a suitable carrier, e.g., bacteria, the phage displays the antibody fragment. Phage display of antibodies may also be provided by combinatorial methods known to those skilled in the art. Antibody fragments displayed by a phage may then be used as part of an immunoassay.

Antibodies against glutathione may be commercially available from Abcam, AbD Serotec, Abgent, Abnova Corporation, ABR-Affinity BioReagents, Acris Antibodies GmbH, Advanced Targeting Systems, Assay Designs/Stressgen Bioreagents, Atlas Antibodies, Aviva Systems Biology, BioGenex, Biosensis, Calbiochem, Cayman Chemical, Epitomics, Inc., Everest Biotech, GeneTex, GenScript Corporation, GenWay Biotech, Inc., HyTest Ltd., IMGENEX, Lab Vision, Lifespan Biosciences, MBL International, Millipore Corporation, Novus Biologicals, ProSci, Inc, Proteintech, Group, Inc., QED Bioscience Inc., R&D Systems, Raybiotech, Inc., Rockland Immunochemicals, Inc., Santa Cruz Biotechnology, Inc., ScyTek Laboratories, and Tocris Bioscience.

In another embodiment, the binding partner may be an aptamer. Aptamers are a class of molecule that represents an alternative to antibodies in term of molecular recognition. Aptamers are oligonucleotide or oligopeptide sequences with the capacity to recognize virtually any class of target molecules with high affinity and specificity. Such ligands may be isolated through Systematic Evolution of Ligands by EXponential enrichment (SELEX) of a random sequence library, as described in Tuerk C, Gold L. Systematic evolution of ligands by exponential enrichment: RNA ligands to bacteriophage T4 DNA polymerase. Science. 1990; 249:505-10. The random sequence library is obtainable by combinatorial chemical synthesis of DNA. In this library, each member is a linear oligomer, eventually chemically modified, of a unique sequence. Possible modifications, uses and advantages of this class of molecules have been reviewed in Jayasena SD. Aptamers: an emerging class of molecules that rival antibodies in diagnostics. Clin Chem. 1999; 45:1628-50. Peptide aptamers consist of conformationally constrained antibody variable regions displayed by a platform protein, such as E. coli Thioredoxin A, that are selected from combinatorial libraries by two hybrid methods (Colas P, Cohen B, Jessen T, Grishina I, McCoy J, Brent R. Genetic selection of peptide aptamers that recognize and inhibit cyclin-dependent kinase 2. Nature. 1996; 380:548-50.).

The binding partners of the invention such as antibodies or aptamers, may be labelled with a detectable molecule or substance, such as a colorimetric, fluorescent or radioactive molecule or any others labels known in the art. Labels are known in the art that generally provide (either directly or indirectly) a signal.

As used herein, the term “labeled”, with regard to the antibody, is intended to encompass direct labeling of the antibody or aptamer by coupling (i.e., physically linking) a detectable substance, such as a radioactive agent or a fluorophore (e.g. fluorescein isothiocyanate (FITC) or phycoerythrin (PE) or Indocyanine (Cy5)) to the antibody or aptamer, as well as indirect labeling of the probe or antibody by reactivity with a detectable substance. An antibody or aptamer of the invention may be labeled with a radioactive molecule by any method known in the art. For example radioactive molecules include but are not limited radioactive atom for scintigraphic studies such as I123, I124, In111, Re186, Re188.

The aforementioned assays generally involve the bounding of the binding partner (ie. Antibody, aptamer or a probe) in a solid support. Solid supports which can be used in the practice of the invention include substrates such as nitrocellulose (e.g., in membrane or microtiter well form); polyvinylchloride (e.g., sheets or microtiter wells); polystyrene latex (e.g., beads or microtiter plates); polyvinylidine fluoride; diazotized paper; nylon membranes; activated beads, magnetically responsive beads, and the like.

More particularly, an ELISA method can be used, wherein the wells of a microtiter plate are coated with a set of antibodies against glutathione. A blood sample containing or suspected of containing glutathione is then added to the coated wells. After a period of incubation sufficient to allow the formation of antibody-antigen complexes, the plate(s) can be washed to remove unbound moieties and a detectably labeled secondary binding molecule added. The secondary binding molecule is allowed to react with any captured sample marker protein, the plate washed and the presence of the secondary binding molecule detected using methods well known in the art.

Glutathione can be determined by Nuclear Magnetic Resonance Spectroscopy.

Measuring the concentration of glutathione may also include separation of the proteins: centrifugation based on the protein's molecular weight; electrophoresis based on mass and charge; HPLC based on hydrophobicity; size exclusion chromatography based on size; and solid-phase affinity based on the protein's affinity for the particular solid-phase that is use. Once separated, glutathione may be identified based on the known “separation profile” e.g., retention time, for that protein and measured using standard techniques. Alternatively, the separated proteins may be detected and measured by, for example, a mass spectrometer.

Another further object of the invention relates to a method for screening an asymptomatic patient at risk for heart failure, said method comprising the steps of:

(i) measuring the concentration of glutathione in a blood sample obtained from said patient,

(ii) comparing the concentration of glutathione measured in step (i) to a control value derived from the concentration of glutathione in blood samples from healthy patients

wherein a decreased concentration of glutathione in the blood sample obtained from said patient as compared to said control value indicates that the patient is at risk for heart failure.

For example, the concentration of glutathione in the blood sample of a patient can be deemed to be decreased when it is less than 2 mM, preferably less than 1.9 mM, even more preferably less than 1.8 mM, 1.7 mM, 1.6 mM or 1.5 mM.

Another further object of the invention relates to a method for classifying a patient at risk for heart failure, wherein said method comprises measuring the concentration of glutathione in a blood sample obtained from said patient.

In a particular embodiment, said method further comprises the steps of:

(i) measuring the concentration of glutathione in a blood sample obtained from said patient,

(ii) comparing the concentration of glutathione measured in step (i) to a reference value derived from the concentration of glutathione in blood samples from patients who are at particular stages of heart failure and/or to a control value derived from the concentration of glutathione in blood samples from healthy patients.

In a further embodiment of the invention, methods of the invention comprise measuring the concentration of at least one further biomarker.

The term “biomarker”, as used herein, refers generally to a molecule, the expression of which in a blood sample from a patient can be detected by standard methods in the art (as well as those disclosed herein), and is predictive or denotes a condition of the subject from which it was obtained.

For example, the other biomarker may be selected from the group heart failure biomarkers consisting of brain natriuretic peptide (BNP), amino-terminal pro-brain natriuretic peptide (NT-pro BNP), norepinephrine, troponin, heart-type fatty acid binding protein, myosin light chain-1, matrix metalloproteinase, tissue inhibitor of matrix metalloproteinase, C-reactive protein (CRP), TNFalpha, sTNFR1, sTNFR2, soluble IL-2 receptor, CD40-CD154, CCAM-I, P-selectin, tissue factor and von Willebrand factor, urocortin, myeloperoxidase, and uric acid.

In a preferred embodiment, the further biomarker of heart failure is NT-pro BNP or sTNFR1.

Yet another object of the invention relates to a kit for performing a method of the invention, said kit comprising means for measuring the concentration of glutathione in a blood sample obtained from a patient. The kit may include means for the performance of the enzymatic methods as described above such as glutathione reductase and DTNB. The kit may alternatively include a probe, an antibody, or a set of antibodies and probes as above described. In a particular embodiment, the antibody or set of antibodies and probes are labelled as above described. The kit may also contain other suitably packaged reagents and materials needed for the particular detection protocol, including solid-phase matrices, if applicable, and standards. The kit may also contain one or more means for the detection of a further biomarker. Typically the kit may also contain means for the detection of one or more heart failure biomarker selected from the group consisting of brain natriuretic peptide (BNP), amino-terminal pro-brain natriuretic peptide (NT-pro BNP), norepinephrine, troponin, heart-type fatty acid binding protein, myosin light chain-1, matrix metalloproteinase, tissue inhibitor of matrix metalloproteinase, C-reactive protein (CRP), sTNFR1, soluble T2 receptor, soluble IL-2 receptor, CD40-CD154, CCAM-I, P-selectin, tissue factor and von Willebrand factor, urocortin, myeloperoxidase, and uric acid.

In a preferred embodiment, kit of the invention comprises means for measuring the concentration of glutathione and means for measuring the concentration of NT-pro BNP and/or sTNFR1.

A further object of the invention relates to the use of blood glutathione as a biomarker for screening an asymptomatic patient at risk for heart failure.

Therapeutic Methods:

The method of the invention may be thus useful for screening or classifying patients at risk for heart failure and then may be used to choose the accurate treatment. For example, patients with a low level of glutathione may receive a more intensive treatment and attention compared to patient with higher level. Such method may thus help the physician to make a choice on a prophylactic treatment, which can accordingly consist in administering accurate drugs to the patients. Costs of the treatments may therefore be adapted to the severity and morbidity of the patients.

As used herein, the term “prevention” refers to preventing the disease or condition from occurring in a subject which has not yet been diagnosed as having it.

Accordingly a further object of the invention relates to a method for preventing heart failure in a patient comprising a step of screening said patient at risk for heart failure according to any method of the invention and a step of administering said patient with the accurate therapeutic drugs and regimen.

For example, drugs that may be useful for the prevention of heart failure may be selected from the group consisting of ACE inhibitors, beta-blockers and aldosterone antagonists.

In a preferred embodiment, the drug is selected as having the capability to restore the glutathione level in the heart. For example, said drug may be glutathione precursors or drugs (N-acetylcysteine, alpha lipoic acid, S-adenosyl-methionine, curcumin), or cysteine precursors (L-2-oxothiazolidine-4-carboxylate), or dietary supplementations (cysteine and glycine) having the capability to restore glutathione level in the heart tissue, such as N-acetylcysteine (NAC) which has the formula of:

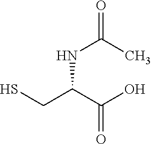

Accordingly, a further object of the invention relates to N-acetylcysteine (NAC) for the prevention of heart failure in a patient wherein said patient has been screened or classified according to one of the methods described above.

In a particular embodiment, the invention relates to a method for preventing heart failure in a patient, said method comprising the steps of:

i) screening or classifying said patient at risk for heart failure according to any method of the invention

ii) administering said patient with an effective amount of NAC.

In a particular embodiment, the patient is a LMNA (Lamin A/C gene) mutated patient.

By an “effective amount of NAC” is meant a sufficient amount of NAC to prevent heart failure at a reasonable benefit/risk ratio applicable to any medical treatment. It will be understood, however, that the total daily usage of NAC will be decided by the attending physician within the scope of sound medical judgment.

The specific therapeutically effective dose level for any particular patient will thus depend upon the severity of the disorder. Other factors may will also impact the therapeutically dose level such as the specific composition employed, the age, body weight, general health, sex and diet of the patient; the time of administration, route of administration, and rate of excretion of NAC; the duration of the treatment; drugs used in combination or coincidental with NAC; and like factors well known in the medical arts. For example, it is well within the skill of the art to start doses of the compound at levels lower than those required to achieve the desired therapeutic effect and to gradually increase the dosage until the desired effect is achieved.

Finally, methods of the invention may be applied for monitoring the therapeutic outcome of a patient who has been screened or classified at risk for heart failure. Accordingly, the present invention relates to a method for monitoring the treatment of patient affected with who has been screened or classified at risk for heart failure, said method comprising the steps consisting of:

i) classifying said patient at risk for heart failure before said treatment by performing the method of the invention

ii) classifying said patient at risk for heart failure after said treatment by performing the method of the invention

iii) and comparing the risk predicted a step i) with the risk predicted at step ii) wherein a difference between said outcomes is indicative of the effectiveness of the treatment.

The invention will be further illustrated by the following figures and examples. However, these examples and figures should not be interpreted in any way as limiting the scope of the present invention.

### BRIEF DESCRIPTION OF SEVERAL VIEWS OF THE DRAWINGS

FIG. 1: Relation between LVEF or blood sTNFR1 level and functional NYHA class in patients undergoing cardiac surgery (n=76) and healthy controls (n=15). The cohort displayed a standard relation between LVEF (A) or sTNFR1 (B) and functional NYHA class. sTNFR1 indicated the cleaved extracellular domain of TNFR1. LVEF: left ventricular ejection fraction. Linear trends P<0.0001. *P<0.05 vs healthy controls are denoted in the figure as “C”. †P<0.05 vs NYHA class I; ‡P<0.05 vs NYHA class II; §P<0.05 vs NYHA class III.

FIG. 2: Relation between atrial tissue glutathione and functional NYHA class in patients undergoing cardiac surgery. Atrial tissue glutathione content was significantly decreased in symptomatic NYHA class IV patients compared with asymptomatic NYHA class I patients. Linear trend P<0.03. *P<0.05 vs NYHA class I.

FIG. 3: Atrial tissue glutathione in the subgroups of patients with coronary artery diseases (CAD) or aortic stenosis (AS), according to preserved LVEF (>45%) or LV dysfunction (45%). (A) Deficiency in atrial glutathione was related with LV dysfunction in CAD patients. In contrast, atrial glutathione was low in AS patients, independently of the LVEF value. *P<0.05 vs LVEF>45%. (B) In CAD patients, atrial glutathione correlated significantly with the LVEF value (r=0.45, P=0.006).

FIG. 4: Blood glutathione in patients undergoing cardiac surgery and in the CAD and AS subgroups of patients. (A) In patients undergoing cardiac surgery, blood glutathione decreased as a function of NYHA class (linear trend P<0.0001). (B) Compared with healthy controls, blood glutathione in the CAD and AS subgroups of patients was depleted, independently of the LVEF value. *P<0.05 vs healthy controls are denoted in the figure as “C”; †P<0.05 vs NYHA class I.

FIG. 5: Correlation between blood glutathione and blood sTNFR1 in patients undergoing cardiac surgery. The decrease in blood glutathione was exponentially related with the elevation in blood sTNFR1 (r=0.88).

FIG. 6: Blood glutathione level is positively correlated with LVEF, S_{T} and S_{M} in LMNA-mutated patients. A) LVEF, measured by standard echocardiography, represented LV systolic function (r=0.44; p=0.03). B) The tricuspid annular velocity, S_{T}, measured by TDE illustrated RV contractility (r=0.53; p=0.01; closed squares); S_{M} measured by TDE illustrated LV contractility (r=0.51; p=0.04; open squares).

FIG. 7: Receiver-operator characteristic curve (ROC) for glutathione in the diagnosis of depressed LVEF or reduced LV/RV contractility in LMNA-mutated patients. A) ROC analysis for glutathione compared patients with depressed LVEF (<50%) to patients with preserved LVEF; Area Under Curve (AUC)-95% CI: 0.83 [0.64-1.01]; B) ROC analysis for glutathione compared patients with reduced S_{M} (<7.5 cm/s) or reduced S_{T} (<11.5 cm/s) to subjects with preserved S_{M} and S_{T}; AUC-95% CI: 0.79 [0.57-1.00].

### EXAMPLES

### Example 1

### Blood Glutathione Deficiency: A Marker of Asymptomatic Cardiac Diseases

Material & Methods:

Patients: The study included 76 patients undergoing cardiac surgery (coronary artery bypass grafting (CABG), aortic valve replacement, orthotopic heart transplantation and ventricular assist device implantation) from 2004 to 2007. Patients with sepsis, endocarditis and renal failure were excluded. Fifteen healthy volunteers with normal left ventricular ejection fraction (LVEF) were recruited by the Centre d'Investigation Clinique of the Hopital Henri Mondor. Clinical data and transthoracic echocardiographies (Vivid 7, GE, Norway), using American Society of Echocardiography recommendations for the measurement of LVEF by two-dimensional echocardiography (Schiller N B, Shah P M, Crawford M, DeMaria A, Devereux R, Feigenbaum H, Gutgesell H, Reichek N, Sahn D, Schnittger I, et al. American Society of Echocardiography Committee on Standards, Subcommittee on Quantitation of Two-Dimensional Echocardiograms. J Am Soc Echocardiogr. 1989; 2:358-67.) were obtained for all patients. Blood samples were obtained from the 15 volunteers. Venous blood samples and right atrial appendages were obtained from patients undergoing cardiac surgery for coronary artery bypass graft or aortic valve replacement with cardiopulmonary bypass. Blood samples only were obtained from patients undergoing left ventricular assist device implantation. Right atrial and venous blood samples were immediately frozen in liquid nitrogen, and stored at −80° C. until use. Paroxystic post-surgery atrial fibrillation was recorded.

All patients had given written informed consent before surgical procedures were performed. All studies are conformed to the Declaration of Helsinki and institutional ethical regulations.

Assays for glutathione and sTNFR1: Atrial tissue samples were cut into 20 μm sections. Homogenates were prepared from 5 frozen sections of each sample by homogenization at 4° C., in 200 μl of 50 mM Hepes, pH 7.4, containing protease inhibitors (1 mM PMSF, 2 μg/ml leupeptin, 2 μg/ml aprotinin), using a tissuelyzer (Qiagen). Glutathione was assayed in atrial homogenates or whole blood, according to a modification of Tietze's method (Tietze F. Enzymic method for quantitative determination of nanogram amounts of total and oxidized glutathione: applications to mammalian blood and other tissues. Anal Biochem. 1969; 27:502-22.) as previously described in Bourraindeloup M et al. (Bourraindeloup M, Adamy C, Candiani G, Cailleret M, Bourin M C, Badoual T, Su J B, Adubeiro S, Roudot-Thoraval F, Dubois-Rande J L, Hittinger L, Pecker F. N-acetylcysteine treatment normalizes serum tumor necrosis factor-alpha level and hinders the progression of cardiac injury in hypertensive rats. Circulation. 2004; 110:2003-9.) and as recently updated (Rahman I, Kode A, Biswas S K. Assay for quantitative determination of glutathione and glutathione disulfide levels using enzymatic recycling method. Nat Protoc. 2006; 1:3159-65.). sTNFR1 was quantified in whole blood with ELISA kits (Quantikine, R&D Systems).

Statistical analysis: Results are given as means±sem. Data were analyzed by Mann-Whitney test or Kruskal-Wallis test and Dunn post test, as appropriate (Prism, GraphPad Software Inc). Differences were considered statistically significant at P<0.05.

Results:

Clinical and biological characteristics of the patients: The mean age of healthy controls was 52±4 years and their mean LVEF was 62±1%. The clinical and biological characteristics of the 76 patients undergoing surgery for coronary artery disease (CAD; n=43), aortic valve stenosis (AS; n=25), or dilated cardiomyopathy (n=8, transplantation or mechanical assist device implantation) are reported in Table 1.

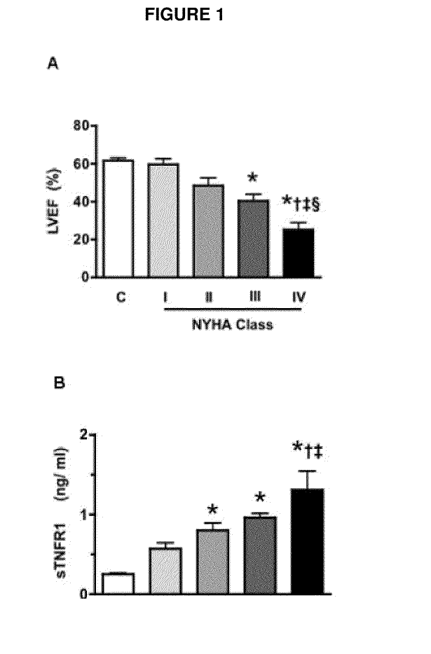

The cohort consisted of 22%, 31%, 29% and 18% patients divided into functional NYHA class I, II, III and IV, respectively. Symptomatic patients in NYHA class III and IV displayed a significant decrease in LVEF compared with the control group, whereas asymptomatic patients of NYHA class I had preserved LVEF (FIG. 1A). Patients with CAD and patients with AS constituted the two principal groups of our cohort. Compared with CAD patients, AS patients displayed significant hypertrophy of septal (ST) and posterior (PWT) walls and high LVEF, illustrating an aortic stenosis-induced cardiomyopathic remodelling supporting compensation of LVEF (Table 1).

Mean blood level of sTNFR1 (a recognized marker of heart failure severity) increased by 3- to 5-fold in symptomatic patients of NYHA class II to IV as compared with healthy controls (FIG. 1B). Of note, the mean blood sTNFR1 level in our asymptomatic, NYHA class I patients, was not statistically different from control values.

TABLE 1
Clinical and biological characteristics of patients undergoing
cardiac surgery

| | | Coronary Artery | Aortic | P Value |
|---|---|---|---|---|
| | All patients | Diseases (CAD) | Stenosis (AS) | CAD |
| | (n = 76) | (n = 43) | (n = 25) | vs AS |
| Demographic | | | | |
| Male/female (n) | 60/16 | 37/6 | 15/10 | 0.01 |
| Age, yrs | 66 ± 1 | 62 ± 2 | 75 ± 2 | <0.0001 |
| NYHA, mean | 2.3 ± 0.1 | 2.2 ± 0.2 | 2.6 ± 0.2 | NS |
| I, (%) | 22 | 31 | 5 | |
| II, (%) | 31 | 31 | 42 | |
| III, (%) | 29 | 26 | 37 | |
| IV, (%) | 18 | 12 | 16 | |
| Clinical characteristics | | | | |
| Hypertension, (%) | 56 | 64 | 36 | NS |
| Hypercholesterolemia, (%) | 56 | 81 | 47 | 0.03 |
| Diabetes mellitus, (%) | 35 | 45 | 17 | NS |
| Chronic Arrythmia, (%) | 12 | 5 | 24 | 0.01 |
| Echocardiographic data | | | | |
| LVEF (%) | 45.3 ± 2.1 | 45 ± 2 | 53 ± 4 | 0.06 |
| LVEDD (mm) | 54.3 ± 1.3 | 55.4 ± 1.5 | 50.1 ± 1.8 | 0.04 |
| iLVEDD (mm · cm⁻²⁾ | 30 ± 1 | 30 ± 1 | 29 ± 1 | NS |
| ST (mm) | 11.6 ± 0.4 | 10.3 ± 0.4 | 13.3 ± 0.7 | 0.0001 |
| PWT (mm) | 10.8 ± 0.4 | 9.8 ± 0.4 | 12.1 ± 0.6 | 0.002 |
| LA diameter (mm) | 41 ± 2 | 41 ± 2 | 42 ± 3 | NS |
| Systolic PAP (mm Hg) | 44 ± 2 | 42 ± 4 | 43 ± 3 | NS |
| Medications (%) | | | | |
| Beta-blockers | 51 | 71 | 22 | 0.0005 |
| ACE inhibitors | 35 | 50 | 11 | NS |
| AT-II type 1 receptor antagonists | 21 | 20 | 22 | NS |
| Diuretics | 48 | 43 | 57 | NS |
| Aldosterone antagonists | 16 | 20 | 9 | NS |
| Statin | 60 | 77 | 35 | 0.001 |
| Surgery characteristics | | | | |
| No urgent surgery, n (%) | 83 | 81 | 100 | 0.02 |
| Post Operative AF (%) | 30 | 21 | 53 | 0.0006 |
| Biochemical data | | | | |
| CRP (mg/l) | 11 ± 2 | 11.4 ± 2.6 | 10.9 ± 6.0 | NS |
| Haemoglobin (g/dl) | 13.1 ± 0.2 | 13.3 ± 0.3 | 12.8 ± 0.5 | NS |
| Total bilirubin (mg/dl) | 17 ± 2 | 17.6 ± 2.6 | 17.4 ± 2.6 | NS |
| Creatinine (μmol/l) | 106 ± 5 | 109 ± 8 | 100 ± 6 | NS |
| Blood glutathione (mM) | 1.4 ± 0.1 | 1.46 ± 0.07 | 1.23 ± 0.06 | 0.02 |
| Tissue glutathione (nmol/mg) | 2.0 ± 0.2 | 2.03 ± 0.23 | 1.91 ± 0.27 | NS |
| Blood sTNF R1 (ng/ml) | 0.9 ± 0.5 | 0.79 ± 0.07 | 0.97 ± 0.07 | NS |

LVEF: left ventricular ejection fraction;
LVEDD: left ventricular end diastolic diameter;
iLVEDD: indexed LVEDD;
ST: end-diastolic septal wall thickness;
PWT: end-diastolic posterior wall thickness;
LA diameter: left atrial diameter;
PAP: pulmonary artery pressure.
CRP: C-reactive protein.
Post-operative AF: post-operative atrial fibrillation. Post-operative AF excluded patients who were in chronic atrial fibrillation, and patient having heart transplant or left ventricular assist device. In CAD and AS, blood glutathione was not correlated with the age (P = 0.46).

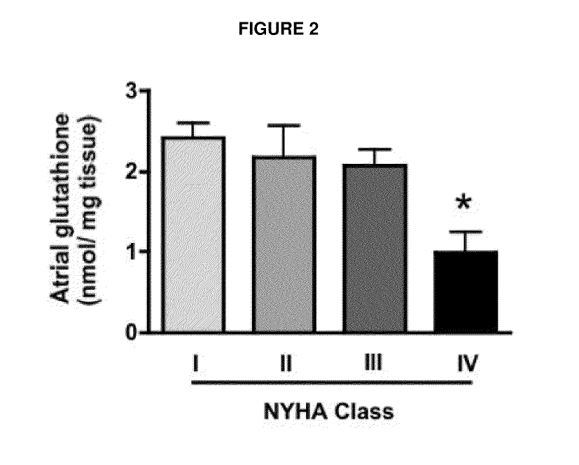

Right atrial Glutathione in patients with heart disease: The mean content of glutathione in right atrial tissue was markedly decreased by 58% in patients of NYHA class IV compared with asymptomatic patients of NYHA class I (1.0±0.2 vs 2.4±0.2 nmol glutathione/mg tissue, respectively; P=0.002; FIG. 2).

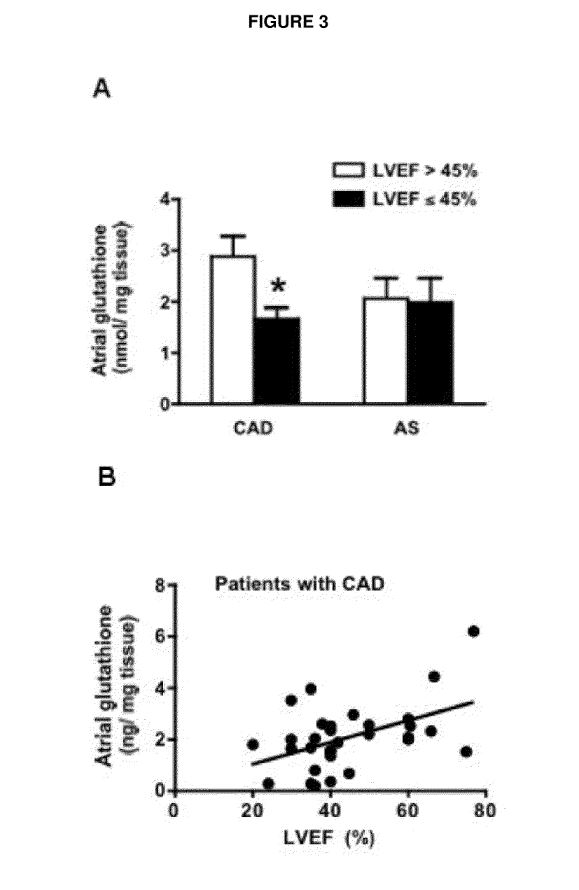

Next, we considered separately the two principal groups of CAD and AS patients, distributed into 2 subgroups on the basis of their LV function. The subgroup of CAD patients with LVEF dysfunction (≦45%) displayed 40% larger deficiency in atrial glutathione content than the subgroup with preserved LVEF (>45%) (FIG. 3A). In fact, in CAD patients, atrial glutathione was positively correlated with the LVEF (r=0.45, P=0.0064; FIG. 3B). In contrast, in AS patients, atrial glutathione content was low independently of the LV function, similar to that of CAD patients with LVEF dysfunction (2.0±0.3 vs 1.7±0.2 glutathione nmol/mg tissue) (FIG. 3A).

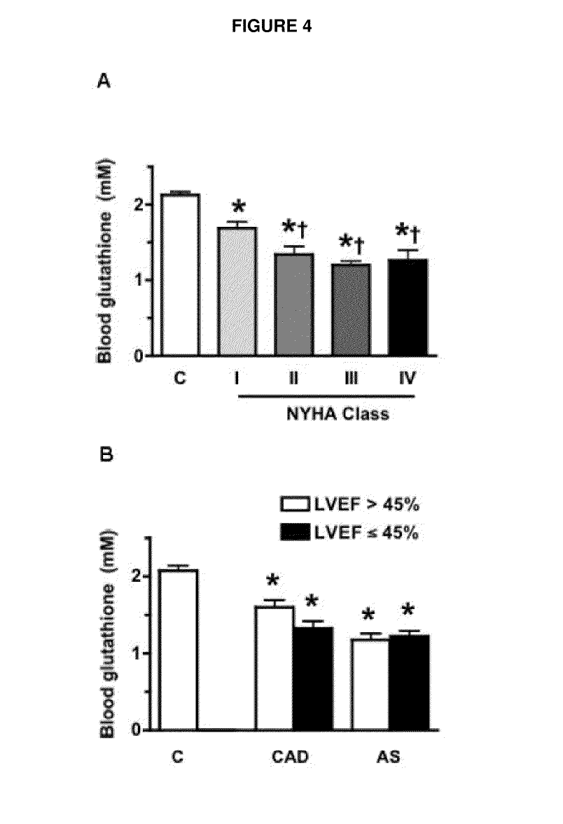

Venous blood glutathione deficiency in patients with cardiac disease: Next, we examined whether deficiency in systemic glutathione affected patients with cardiac diseases. Considering the all cohort of patients, there was no significant correlation between the right atrial and venous blood glutathione concentrations (r=0.11, P=0.43). Nevertheless, as compared with healthy controls, mean venous blood level of glutathione was significantly decreased in patients with cardiac diseases (from 20% in NYHA class I to 40% in NYHA class II to IV). And mean venous blood levels of symptomatic patients of NYHA class II to IV were significantly lower than that of asymptomatic patients of NYHA class I (p<0.05, FIG. 4A). Venous blood glutathione levels in CAD and AS patients were significantly lower than that of healthy controls, independently of the LVEF value (FIG. 4B).

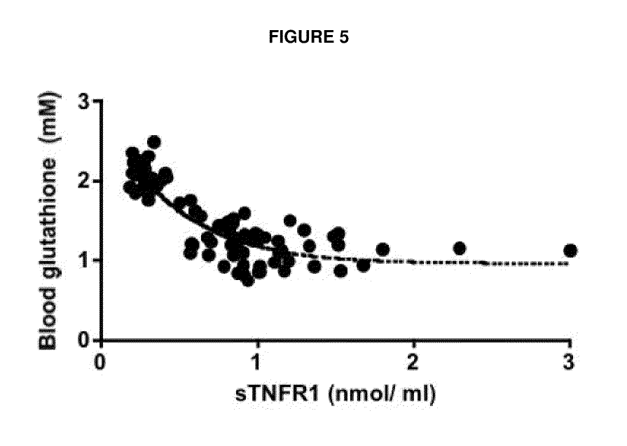

Finally, the decrease in blood glutathione level was exponentially correlated with the increase in blood sTNFR1 level (r=0.88, FIG. 5).

Conclusion:

The first important finding of this study is that a drop in blood glutathione discriminates asymptomatic NYHA I class patients from healthy controls. This observation brings to light the potential of blood glutathione test for screening asymptomatic individuals at risk for heart failure. As previously stated (Goldberg L R, Jessup M. Stage B heart failure: management of asymptomatic left ventricular systolic dysfunction. Circulation. 2006; 113:2851-60.), an improved identification of asymptomatic individuals, through screening and diagnosis, may lead to the early initiation of appropriate pharmacological therapy. Appropriate therapy, in turn, can improve outcomes and decrease progression to heart failure, morbidity and mortality.

Optimized screening of asymptomatic patients will rely on the combination of independent biomarkers ahead of clinical examination. For example, blood glutathione test can be combined with NT-proBNP. The interest of such a combination relies on the independent information provided by each marker, the one related with vascular injury, and the other one related with oxidative stress and inflammation. The predictive value of the combination of two such markers is much greater than that of each biomarker taken separately.

These data further document the potentiality of oral administration of a glutathione precursor, as for example N-acetylcysteine (NAC), as a complement treatment to current medical therapies for cardiac diseases. Noteworthy, glutathione deficiency will not only weaken cell defence against oxidative stress, but also many other cellular functions including cell survival. Interestingly, a recent study Ozaydin et al. reported that NAC, administered by intravenous infusion for 1 h before and 48 h after cardiac surgery, decreased the incidence of postoperative atrial fibrillation (AF) (Ozaydin M, Peker O, Erdogan D, Kapan S, Turker Y, Varol E, Ozguner F, Dogan A, Ibrisim E. N-acetylcysteine for the prevention of postoperative atrial fibrillation: a prospective, randomized, placebo-controlled pilot study. Eur Heart J. 2008; 29:625-31.). The inventors have discovered that NAC administration to an asymptomatic patient at risk for heart failure can be optimized by performing the method for screening and/or classifying said asymptomatic patient prior to treatment.

In conclusion, our findings bring to light the systemic deficiency in glutathione in asymptomatic patients with structural heart disease. Accordingly, blood glutathione provides a new biomarker for screening asymptomatic patients at risk for heart failure.

### Example 2

### Glutathione in LMNA-Mutated Patients

Introduction:

Lamin A/C proteins form an organized meshwork between the inner nuclear membrane and the chromatin that is essential for the maintenance of nuclear structure and functions. Mutations of the LMNA gene have been causally related to a variety of diseases, the majority with cardiac phenotypes characterized by atrial fibrillation, conduction system disease requiring pacemaker implantation, sudden death and heart failure. We have previously reported that life-threatening ventricular arrhythmias could be adequately prevented by prophylactic internal cardioverter defibrillator (ICD) implantation. Apart from ventricular arrhythmias, heart failure requires prompt detection and management in LMNA-mutated patients, as by age 50, over 60% of these patients have overt symptoms of heart failure. We have recently reported that NT-proBNP accurately detected reduced contractility in LMNA-mutated patients.

The tripeptide glutathione (L-gamma-glutamyl-cysteinyl-glycine) is the most abundant thiol/disulphide component of the eukaryotic cell, hence a key player in cell defense against oxidative stress that also serves vital functions, being essential for vascular and cardiac function. Glutathione deficiency and oxidative stress promotes inflammation and the production of the pro-inflammatory cytokine tumor necrosis factor alpha (TNF). High blood levels of TNF and of the cleaved domain of its type-1 receptor, sTNFR1, are mortality predictors in patients with heart failure.

In a previous report we pointed out that systemic glutathione deficiency characterized patients with structural or functional cardiac abnormalities. In addition, in a mouse model carrying a missense LMNA mutation and developing a dilated cardiomyopathy, we found a systemic glutathione deficiency in symptomatic animals. In this experimental model, oral treatment with the glutathione precursor, N-acetylcysteine (NAC), significantly improved heart structure and function, which suggested a causal relationship between glutathione deficiency and the progression of the cardiac disease.

The present prospective study aimed to explore systemic glutathione and STNFR1 status in LMNA-mutated subjects, and to determine the possible value of each marker to diagnose or to characterize the stage of the cardiac disease.

Methods:

Patients: The present study, carried out prospectively for a period of 25 months (April 2007 and April 2009) at the AP-HP, Groupe Hospitalier Cochin, Service de Cardiologie, Paris, France, enrolled 23 patients with LMNA gene mutation referred to our institution for cardiac evaluation. All patients underwent a physical examination, an echocardiography, and biological tests including the measurements of whole blood glutathione and whole blood sTNFR1. Blood levels of glutathione and sTNFR1 in patients were compared to those of a group of 15 healthy volunteers including 8 males and 7 females, ranging in age from 30 to 70 years.

Echocardiographic measurements: Physicians, blinded to the biological results, performed standard echocardiographic examinations that were implemented by pulsed tissue-Doppler echocardiography (TDE), using an ATL HDI 5000 system (ATL Ultrasound, Bothell, Wash.). Examinations conformed to the recommendations of the American Society of Echocardiography. Left ventricular ejection fraction (LVEF) was determined according to the Simpson's method; a LVEF<50% is considered as reduced LVEF in our Echo-Laboratory. Mean (lateral and septal) systolic mitral annular velocity (S_{M}) and tricuspid systolic annular velocity (S_{T}) were evaluated by TDE to assess LV and RV contractility, respectively. Reduced LV contractility was defined as S_{M}<7.5 cm/s.

Assays for glutathione and sTNFR1: With the patient at rest, 10 ml blood samples were collected in tubes containing ethylene diamine tetraacetic acid. The samples were immediately stored at −80° C. until used. Whole blood glutathione was measured according to a modification of Tietze's recycling assay, as previously used (Adamy C, Mulder P, Khouzami L, Andrieu-Abadie N, Defer N, Candiani G, et al. Neutral sphingomyelinase inhibition participates to the benefits of N-acetylcysteine treatment in post-myocardial infarction failing heart rats. J Mol Cell Cardiol 2007; 43:344-53; Damy T, Kirsch M, Khouzami L, Caramelle P, Le Corvoisier P, Roudot-Thoraval F, et al. Glutathione deficiency in cardiac patients is related to the functional status and structural cardiac abnormalities. PLoS ONE 2009; 4:e4871.; Bourraindeloup M, Adamy C, Candiani G, Cailleret M, Bourin M C, Badoual T, et al. N-acetylcysteine treatment normalizes serum tumor necrosis factor-alpha level and hinders the progression of cardiac injury in hypertensive rats. Circulation 2004; 110:2003-9. Cailleret M, Amadou A, Andrieu-Abadie N, Nawrocki A, Adamy C, Ait-Mamar B, et al. N-acetylcysteine prevents the deleterious effect of tumor necrosis factor-(alpha) on calcium transients and contraction in adult rat cardiomyocytes. Circulation 2004; 109:406-11) and as recently updated by Rahman et al. (Rahman I, Kode A, Biswas S K. Assay for quantitative determination of glutathione and glutathione disulfide levels using enzymatic recycling method. Nat Protoc 2006; 1:3159-65.). In brief, it is a spectrophotometric/microplate reader assay method, relying on oxidation of reduced glutathione (GSH) by the sulfhydryl reagent 5,5′-dithio-bis(2-nitrobenzoic acid) (DTNB) to form the yellow derivative 5′-thio-2-nitrobenzoic acid (TNB), measurable at 405 nm. Glutathione disulfide (GSSG) is recycled to GSH by glutathione reductase in the presence of NADPH. This method is simple, convenient, sensitive, accurate and rapid, and can assay glutathione in whole blood and tissues. In addition, it uses sulfosalicylic acid for sample preparation, which inhibits gamma-glutamyl transferase and limits glutathione loss. Whole blood sTNFR1 was quantified with ELISA kits (Quantikine, R&D Systems).

Statistical analysis: The data are expressed as mean±s.e.m for continuous variables, and number and percentage for categorical variables. Mann-Whitney test was used for comparisons of continuous variables, and Chi-square test or Fisher exact test for differences in frequency, as appropriated. To investigate the possible influence of various characteristics in relationship with glutathione or sTNFR1 concentrations, single variable analysis were performed. Correlation of glutathione or sTNFR1 with echocardiographic and clinical indexes was assessed using Pearson's correlation coefficient.

We also evaluated the predictive value of blood glutathione level to detect depressed LVEF (<50%), or reduced contractility (S_{M}<7.5 cm/s or S_{T}<11.5 cm/s) by constructing receiver operating characteristics (ROC) curves relating each functional parameter to blood glutathione. We determined the sensitivity, specificity, positive and negative predictive values of glutathione using a cut-off value of 1.835 mM that was previously defined as the optimal cut-off value to discriminate between healthy controls and asymptomatic patients of NYHA class 1 with structural cardiac abnormalities Damy T, Kirsch M, Khouzami L, Caramelle P, Le Corvoisier P, Roudot-Thoraval F, et al. Glutathione deficiency in cardiac patients is related to the functional status and structural cardiac abnormalities. PLoS ONE 2009; 4:e4871.).

P<0.05 was considered statistically significant (STATA10.0 statistical software, StataCorp LP, College Station, Tex.).

Results:

Clinical characteristics of LMNA-mutated patients: Twenty-three consecutive patients with LMNA mutations were enrolled, including 13 related patients from 3 separate families, and 10 unrelated patients. Baseline characteristics of these patients are summarized in Table 2.

TABLE 2
Baseline characteristics of LMNA-mutated patients
(for additional details see Meune et al.(10)).

| | |
|---|---|
| Men/women (n) | 10/13 |
| Age in years | 39 ± 4 |
| Muscular phenotype (n) | |
| Dilated cardiomyopathy + conduction system disease | 8 |
| Emery-Dreifuss muscular dystrophy | 8 |
| Limb-girdle muscular dystrophy | 2 |
| Absence of muscular involvement | 5 |
| Past supra-ventricular arrhythmias (n) | 9 |
| Spontaneous/inducible ventricular arrhythmias (n) | 5 |
| ICD implantation (n) | 7 |
| Shock delivered by ICD (n) | 3 |
| NYHA functional class I/II (n) | 18/5 |
| Treatment (n) | |
| Angiotensin Converting Enzyme Inhibitor | 8 |
| Beta-blocker | 10 |
| Heart rate (beat/min) | 72 ± 2 |
| Systolic blood pressure (mm Hg) | 115. ± 2 |
| Diastolic blood pressure (mm Hg) | 65 ± 1 |
| LVEF (%) | 59 ± 2 |

ICD: internal cardioverter defibrillator;
NYHA: New York Heart Association functional class;
LVEF: left ventricular ejection fraction.

The mean age was 39±4 years. The primary clinical presentation was as follows: 8 patients presented with Emery-Dreifuss muscular dystrophy, 8 with dilated cardiomyopathy with cardiac conduction system disease, 2 with limb-girdle muscular dystrophy; the remaining 5 patients were screened as family members of the probands. Only 3 patients were wheel-chaired; the remaining had no or limited muscle weakness. Nine patients had previous supra-ventricular arrhythmia; all were in sinus rhythm at baseline. Seven patients had undergone ICD implantation. Only 4 patients had reduced LVEF whereas a reduced contractility was demonstrated in 8 patients using TDE.

Blood glutathione deficiency in LMNA-mutated patients is related to NYHA functional class: Blood sTNFR1 level in LMNA-mutated patients showed a trend towards increase compared to healthy controls, but failed to discriminate between the two groups (0.30±0.03 vs 0.25±0.01 ng/ml, ns p=0.18; Table 3), and was markedly elevated (>0.4 ng/ml) in only 2 out of 5 patients of NYHA class II. In contrast, blood glutathione was significantly lower in LMNA-mutated patients than in healthy controls (1.88±0.07 vs 2.13±0.04 mM, p=0.007). Glutathione deficiency was further aggravated in patients of NYHA class II as compared to class I patients (28% vs 8% below the control value, p=0.016, Table 3).

TABLE 3
Blood glutathione and sTNFR1 in LMNA-mutated patients
compared to healthy volunteers

| | | LMNA-mutated Patients | | |
|---|---|---|---|---|
| | Controls | All | NYHA I | NYHA II |
| Variables | (n = 15) | (n = 23) | (n = 18) | (n = 5) |
| sTNFR1 | 0.25 ± 0.01 | 0.30 ± 0.03 | 0.28 ± 0.02 | 0.38 ± 0.12 |
| (pg/ml) | | | | |
| Glutathione | 2.13 ± 0.04 | 1.88 ± 0.07# | 1.97 ± 0.08## | 1.54 ± |
| (mM) | | | | 0.10###† |

#, ##, ### p = 0.007, 0.053 and 0.001 vs healthy volunteers;
†p = 0.016 vs NYHA class I patients.

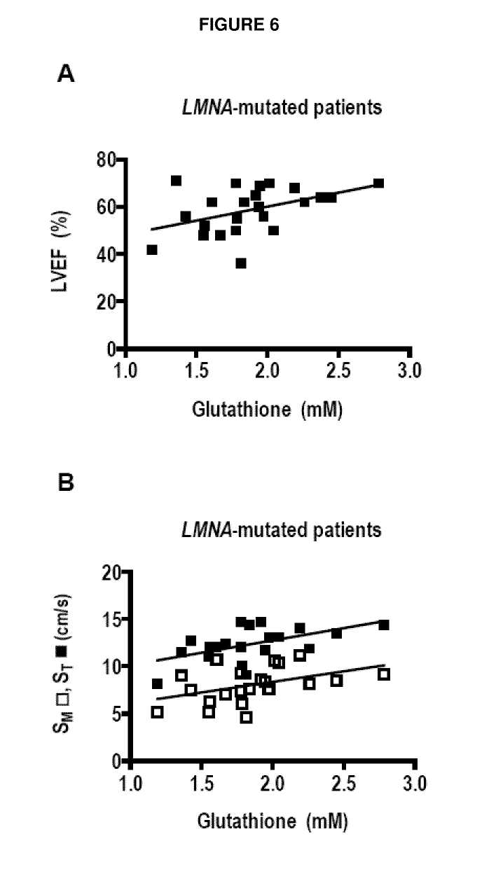

Blood glutathione deficiency in LMNA-mutated patients is related to an altered cardiac function: Next, to analyze the global cardiac function in LMNA-mutated patients in relation to blood levels of sTNFR1 or glutathione, we considered 3 parameters: 1) LVEF measured by standard echocardiography, 2) S_{M} and 3) S_{T}, measured by TDE as sensitive markers of LV and RV contractility, respectively. Reduced cardiac function was defined as the composite index {LVEF<50% or S_{M}<7.5 cm/s or S_{T}<11 cm/s}. Cutoff values, 0.33 ng/ml for blood sTNFR1 and 1.835 mM for blood glutathione, were those that we previously determined as being optimal to discriminate between healthy controls and NYHA class I-IV cardiac patients (Damy T, Kirsch M, Khouzami L, Caramelle P, Le Corvoisier P, Roudot-Thoraval F, et al. Glutathione deficiency in cardiac patients is related to the functional status and structural cardiac abnormalities. PLoS ONE 2009; 4:e4871). The distribution of the patients with altered global cardiac function according to high or low (FIG. 6A left), or increasing level of blood sTNFR1 (FIG. 6A right) was unpredictable, demonstrating the absence of correlation between the two parameters. Conversely, high blood glutathione was associated with preserved cardiac function. Thus, 73% of the patients with blood glutathione<1.835 mM had a depressed cardiac function, whereas 92% of the patients with blood glutathione≧1.835 mM showed a normal cardiac function. The distribution of the patients according to glutathione blood level further pointed out that all the patients with glutathione≧2.3 mM had a normal cardiac function. This distribution was corroborated by the positive correlation between blood glutathione level and each of the 3 parameters: LVEF (r=0.44, p=0.032; FIG. 6A), S_{T} (r=0.53, p=0.010; FIG. 6B) and S_{M} (r=0.51, p=0.046; FIG. 6B).

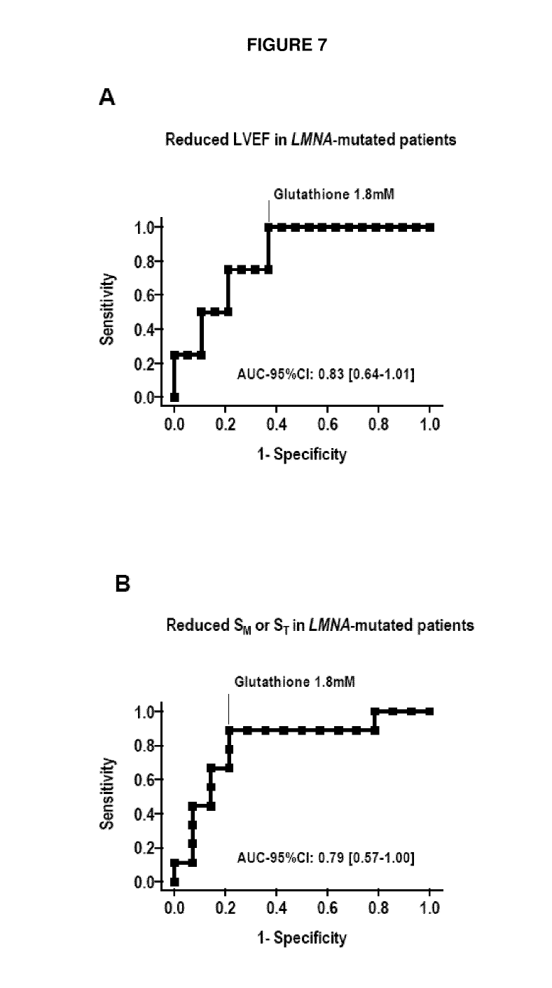

The performance characteristics of glutathione for identifying LMNA-mutated patients with depressed LVEF (<50%), or reduced cardiac LV or RV contractility defined by the composite index {S_{M}<7.5 cm/s or S_{T}<11.5 cm/s}, were re-assessed considering patients with normal LVEF, or normal cardiac contractility, as controls. Under this condition, ROC curve analysis demonstrated blood glutathione as a good indicator of either depressed LVEF (Area Under Curve (AUC)-95% CI: 0.83 [0.64-1.01]; FIG. 7A) or reduced contractility (AUC-95% CI: 0.79 [0.57-1.00]; FIG. 7B). Sensitivity and specificity of the blood glutathione test, obtained with <1.835 mM as the cutoff value, are reported in Table 4. It gave 100% sensitivity and 63% specificity for diagnosing depressed LVEF, 89% sensitivity and 75% specificity for diagnosing reduced contractility. Of note, ROC curve analysis validated the 1.8 mM cut-off value for glutathione (FIGS. 7 A and B). Lastly, a strategy based on NT-proBNP assay and glutathione test appeared to be highly sensitive to detect reduced contractility (Table 4).

TABLE 4
Sensitivity, specificity, and positive (PPV) and negative (NPV)
predictive values of blood glutathione <1.835 mM alone, or
in combination with NT-proBNP ≧125 pg/ml, in the detection
of depressed LVEF or reduced cardiac function.

| | Sensitivity | Specificity | PPV | NPV |
|---|---|---|---|---|
| Glutathione <1.835 mM | | | | |
| Depressed LVEF | 100% | 63% | 36% | 100% |
| Reduced S_{M} or S_{T} | 89% | 75% | 73% | 90% |
| Glutathione <1.835 mM or | | | | |
| NT-proBNP ≧125 pg/ml | | | | |
| Depressed LVEF | 100% | 44% | 29% | 100% |
| Reduced S_{M} or S_{T} | 100% | 58% | 62% | 100% |

LVEF was measured by standard echocardiography, and considered as depressed when <50%. Mean (lateral and septal) systolic mitral annular velocity (SM) and tricuspid systolic annular velocity (ST) were evaluated by TDE. SM<7.5 cm/s defined reduced LV contractility (Alam M, Wardell J, Andersson E, Samad B A, Nordlander R. Effects of first myocardial infarction on left ventricular systolic and diastolic function with the use of mitral annular velocity determined by pulsed wave doppler tissue imaging. J Am Soc Echocardiogr 2000; 13:343-52.; Pellerin D, Sharma R, Elliott P, Veyrat C. Tissue Doppler, strain, and strain rate echocardiography for the assessment of left and right systolic ventricular function. Heart 2003; 89 Suppl 3:iii9-17; Yu C M, Sanderson J E, Marwick T H, Oh J K. Tissue Doppler imaging a new prognosticator for cardiovascular diseases. J Am Coll Cardiol 2007; 49:1903-14.); ST<11.5 cm/s defined reduced RV contractility (Meluzin J, Spinarova L, Bakala J, Toman J, Krejci J, Hude P, et al. Pulsed Doppler tissue imaging of the velocity of tricuspid annular systolic motion; a new, rapid, and non-invasive method of evaluating right ventricular systolic function. Eur Heart J 2001; 22:340-8.).

Discussion:

The present study gives evidence that systemic glutathione deficiency in patients with LMNA mutation is strongly associated with cardiac involvement.

Dilated cardiomyopathy in LMNA-mutated patients is characterized by high rates of major cardiac events including life-threatening arrhythmias or end-stage heart failure. It occurs rarely in young subjects under 20 years, but its overall penetrance progressively increases with age, exceeding 60% in patients older than 50 years. The challenge to physicians is to ensure an early diagnosis delineating the extent of cardiac involvement in young asymptomatic LMNA-mutated subjects, which will enable efficient lifestyle recommendations and preventive therapeutic strategies. Indeed, benefits of an early management of the cardiac disease have been proven by several clinical trials for asymptomatic patients with LV systolic dysfunction and for patients with Duchenne muscular dystrophy (DMD), another inherited disease. The value of several biomarkers is recognized for the diagnostic and prognostic stratification of heart failure comprising sTNFR1, natriuretic peptides, uric acid. However, biomarkers for the diagnosis of asymptomatic patients at risk for heart diseases, that would help to design preventive strategies, are missing keys.

Our finding that glutathione deficiency occurs at onset of cardiac involvement in LMNA-mutated patients, converges with the previous observation by Charniot et al. (Charniot J C, Bonnefont-Rousselot D, Marchand C, Zerhouni K, Vignat N, Peynet J, et al. Oxidative stress implication in a new phenotype of amyotrophic quadricipital syndrome with cardiac involvement due to lamin A/C mutation. Free Radic Res 2007; 41:424-31.) that oxidative stress in these patients occurs prior to heart failure symptoms, and participates to the progression of the degenerative disease. In contrast, inflammation marked by sTNFR1, appears as a late event in LMNA-mutated patients.

Natriuretic peptides, including the brain natriuretic peptide (BNP) and the N-terminal pro-brain natriuretic peptide (NT-proBNP), are useful markers in the diagnosis and management of heart failure. The capacity of B-type natriuretic peptides to detect cardiac involvement in inherited myopathies has been controversed. We recently demonstrated that NT-proBNP (cutoff value of 125 pg/ml) accurately stratified patients into groups with preserved and reduced contractility in this cohort. Because of the complementary prospects offered by each glutathione and NT-proBNP test, we examined the predictive value of the composite index {glutathione<1.835 mM or NT-proBNP≧125 pg/ml} to diagnose depressed LVEF or reduced cardiac function. Combining glutathione and NT-proBNP tests in LMNA-mutated patients improves the diagnostic accuracy of reduced contractility to 100% sensitivity and 100% NPV (Table 4).

In this study, we investigated cardiac function using echography and pulsed TDE. Previous studies have demonstrated that pulsed TDE is more sensitive than conventional echocardiography to detect reduced contractility. In addition, these TDE indexes have demonstrated prognostic significance. While the objective of glutathione concentration and/or NT-proBNP is to detect cardiac involvement at an early stage, we assume that targeting the detection of reduced TDE indexes is more accurate than reduced LVEF.

Systemic glutathione deficiency reported herein is a common feature to cardiomyopathies of different aetiologies. Several experimental studies, reporting the benefits of treatment with the glutathione precursor N-acetylcysteine (NAC), support the causal relationship between glutathione deficiency and the severity of the cardiac disease. Thus, oral NAC treatment improves cardiac structure and function in heart-failing hypertensive rats, in cardiomyopathic mice with missense LMNA mutation, and reverses established hypertrophy in a mouse model of human hypertrophic cardiomyopathy. In patients, oral NAC treatment prevents post-operative atrial fibrillation.

In conclusion, we have shown that systemic glutathione deficiency in LMNA-mutated patients is related to cardiac involvement. The possible clinical benefits of these findings are twofold. First, glutathione deficiency may be proposed as a test to diagnose cardiac involvement, at a preclinical stage, in LMNA-mutated patients. Secondly, because glutathione deficiency occurs early, and is likely to contribute to the progression of the cardiac disease, our finding paves the way to initiate clinical trials to investigate whether NAC supplementation may delay or prevent cardiac failure in LMNA-mutated patients, and possibly, in patients with other inherited cardiomyopathies.

### REFERENCES

Throughout this application, various references describe the state of the art to which this invention pertains. The disclosures of these references are hereby incorporated by reference into the present disclosure.

1. A method for screening an asymptomatic patient at risk for heart failure, said method comprising measuring the concentration of glutathione in a blood sample obtained from said patient.

2. The method according claim 1, wherein said method further comprises measuring the concentration of at least one biomarker selected from the group consisting of brain natriuretic peptide (BNP), amino-terminal pro-brain natriuretic peptide (NT-pro BNP), norepinephrine, troponin, heart-type fatty acid binding protein, myosin light chain-1, matrix metalloproteinase, tissue inhibitor of matrix metalloproteinase, C-reactive protein (CRP), TNFalpha, soluble tumor necrosis factor receptor 1 (sTNFR1), soluble TNFR2 receptor, soluble IL-2 receptor, CD40-CD154, CCAM-I, P-selectin, tissue factor and von Willebrand factor, urocortin or uric acid.

3. The method according to claim 2, wherein said method further comprises measuring the concentration of NT-pro BNP and/or sTNFR1.

4. The method according to claim 1 wherein said patient is affected with a cardiac and/or vascular disease.

5. The method according to claim 1 wherein said patient is diagnosed with a genetically linked cardiovascular disease, hypertension (high blood pressure), pulmonary hypertension, aortic and mitral valve disease (e.g. stenosis), aortic coarctation, coronary disorders, chronic arrhythmias (e.g. atrial fibrillation), cardiomyopathy of any cause, coronaropathy, valvulopathy or cardiac fibrosis.

6. The method according to claim 1 wherein said patient is at risk for heart failure because of diabetes, obesity, aging, smoking, dyslipidemia, intoxication or a genetic disease.

7. The method according to claim 1 wherein said patient is a LMNA (Lamin A/C gene) mutated patient.

8. A method for classifying a patient at risk for heart failure, wherein said method comprises the steps of:
(i) measuring the concentration of glutathione in a blood sample obtained from said patient,
(ii) comparing the concentration of glutathione measured in step (i) to a reference value derived from the concentration of glutathione in blood samples from patients who are at particular stages of heart failure and/or to a control value derived from the concentration of glutathione in blood samples from healthy patients.

9. A kit for performing a method according to claim 1 wherein said kit comprises means for measuring the concentration of glutathione in a blood sample obtained from the patient.

10. The kit according to claim 9 wherein said kit further comprises means for measuring the concentration of least one biomarkers selected from the group consisting of brain natriuretic peptide (BNP), amino-terminal pro-brain natriuretic peptide (NT-pro BNP), norepinephrine, troponin, heart-type fatty acid binding protein, myosin light chain-1, matrix metalloproteinase, tissue inhibitor of matrix metalloproteinase, C-reactive protein (CRP), TNFalpha, soluble tumor necrosis factor receptor 1 (sTNFR1), soluble T2 receptor, soluble IL-2 receptor, CD40-CD154, CCAM-I, P-selectin, tissue factor and von Willebrand factor or urocortin or uric acid.

11. The kit according to claim 9, wherein said kit comprises means for measuring the concentration of NT-pro BNP and/or sTNFR1.

12. A method for preventing heart failure in a patient, said method comprising the steps of:
i) screening or classifying said patient at risk for heart failure according to any method of claim 8, and
ii) administering said patient with an effective amount of NAC or other drug having the capability to restore tissue glutathione.

13. The method according to claim 12 wherein said patient is a LMNA-mutated patient.

14. A method for monitoring the treatment of patient affected with who has been screened or classified at risk for heart failure, said method comprising the steps consisting of:
i) classifying said patient at risk for heart failure before said treatment by performing the method according to claim 8,
ii) classifying said patient at risk for heart failure after said treatment by performing the method according to claim 8, and
iii) comparing the risk predicted a step i) with the risk predicted at step ii) wherein a difference between said outcomes is indicative of the effectiveness of the treatment.
